(19) United States
(12) Patent Application Publication
(10) Pub. No.: US 2023/0123714 A1
(43) Pub. Date: Apr. 20, 2023

(54) STOP DRUNK

(71) Applicant: Travis A Morgan, Atlanta, GA (US)

(72) Inventor: Travis A Morgan, Atlanta, GA (US)

(21) Appl. No.: 16/733,394

(22) Filed: Jan. 3, 2020

(51) Int. Cl. A61K 8/42; A61K 8/67; A61K 8/9789; A61K 8/99

(52) U.S. Cl. CPC A61K 8/42; A61K 8/673; A61K 8/9789; A61K 8/99

(57) ABSTRACT

“Stop Drunk” is the brainchild of Creative Travis A Morgan.

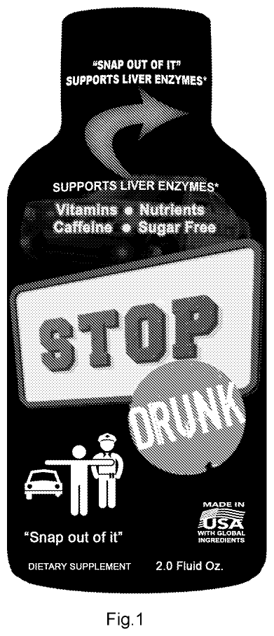

The intent in creating “Stop Drunk”, is to help to relieve, and ultimately deplete the effects of alcohol in the body safely, and at an accelerated rate. The products first offering will be made available as a liquid, dispensed in 1.9 ounce containers, in stores and online, for immediate, single serving consumption.

### BACKGROUND

“Stop Drunk” was designed, and born out of the desire to help to curtail the effects of alcohol consumption, and the often times tragic incidents stemming from it; at a safely accelerated rate. The brainchild of Creative Travis Morgan, the product was formulated by a Physician and a team Bio-chemist. With the sole focus on creating a product that could be consumed conveniently, in a small concentrated dose and most of all, act quickly. Stop Drunk has been four(4) years in the making and literally took shipment of it's first inventory on October of 2021. To date, although rumored that there have been several attempts at creating a product such as Stop Drunk, no product has been successful. Because of some of it's component vitamins and mineral, Amino acids and enzymes; Stop Drunk also excels at comforting the body, vitalizing and re-energizing it. It is the world's 1st scientifically designed product, specifically created to initiate the sobering effects of the body's metabolism of alcohol. By the immediate delivery in a concentrated liquid form, a unique combination of enzyme enhancers. Through their natural adaptation to the body's natural enzymes; they begin to sober the subject sober and lowering the Blood Alcohol Content.

### DESCRIPTION

It is the world's 1st scientifically designed product, specifically created to initiate the sobering effects of the body's metabolism of alcohol. By the immediate delivery in a concentrated liquid form, a unique combination of enzyme enhancers. Through their natural adaptation to the body's natural enzymes; the process initiates the body's natural process of metabolizing it's alcohol content. Ultimately sobering the subject and lowering the Blood Alcohol Content. Currently manufactured as a liquid and dispensed in 2.oz plastic bottles, “Stop Drunk” is conveniently and easily consumed. The recommended dosage is One(1) 2 ounce bottle and 12 to 16 ounces of water. Listed below are the active ingredients in “Stop Drunk”, their purpose and effects on the body are listed individually.

### DESCRIPTION CONTINUED

1. Caffeine: The purpose of the caffeine in “Stop Drunk” blend lies in its ability to snap the brain awake and help the consumer to stay mentally alert. This addition to the drink is the equivalent to one good strong cup of coffee.

2. Milk Thistle: This natural purple flowered plant aids the liver in the detoxification process. Since we know alcohol metabolism occurs primarily in the liver, the addition of milk thistle speeds up the process as well as scavenging free radicals and is a known anti-inflammatory.

3. Probiotics: The addition of probiotics to the blend is to help the intestinal system process the alcohol that is consumed and stabilize gut metabolism.

4. Activated Charcoal: This compound absorbs alcoholic toxins that occur when it is ingested and thus aid in the digestion and metabolism of alcohol.

5. The complex of B vitamins included in “Stop Drunk” help combat the diuretic effect of the ingestion of alcohol. In this way they help to preserve the body's overall volume status and avoid the effects of dehydration.

### SUMMARY

In summary, “Stop Drunk” is without a doubt the most viably powerful, culturally significant, and societal shifting product to enter the marketplace in decades! The implications for this product are incredible. When we consider the number of D.U.I.'s and drunk driving accidents that occur in are society every day, “Stop Drunk” is a much welcomed agent in fighting this fight.

Many have lost their jobs, families, professional integrity and yes in many cases their lives due to alcohol consumption. This product was conceptualized, developed and manufactured with sole intent of making a much needed change to the societal landscape when it comes to these issues.

1. “Stop Drunk”'s unique component combination of enzyme enhancers, vitamins and herbal supplements, combine to enhance the livers ability to metabolize alcohol.
